(19) United States
(12) Patent Application Publication
(10) Pub. No.: US 2019/0153605 A1
(43) Pub. Date: May 23, 2019

(54) AQUEOUS ORGANIC ACIDIC SOLUTION FOR STERILIZATION AND MICROBIAL GROWTH INHIBITION

(71) Applicant: John Barclay Carter, Tierra Verde, FL (US)

(72) Inventor: John Barclay Carter, Tierra Verde, FL (US)

(21) Appl. No.: 16/250,797

(22) Filed: Jan. 17, 2019

Related U.S. Application Data
(62) Division of application No. 12/953,924, filed on Nov. 24, 2010, now abandoned.
Application No. 12/953,924 is a continuation of application No. 10/967,751, filed on Oct. 18, 2004, now abandoned.
(60) Provisional application No. 60/517,797, filed on Nov. 5, 2003.

(51) Int. Cl. C23G 1/02; A61K 31/19; A61K 45/06

(52) U.S. Cl. CPC C23G 1/02; A61K 45/06; A61K 31/19

(57) ABSTRACT

The present invention consists of a two acid core aqueous acidic solution with a pH value of less than one that is nonreactive to human skin, can be safely ingested, and is effective as both a disinfectant and a microbial growth inhibition agent. The solution consists of hydrochloric acid admixed aqueous citric acid. The present invention further comprises a method of disinfecting food products and related surfaces during processing, preparation, and transport.

### CROSS-REFERENCE TO RELATED APPLICATIONS

This application claims priority to U.S. nonprovisional patent application Ser. No. 10/967,751 entitled “GRAS two acid low PH compound consisting of citric and hydrochloric or phosphoric acids filed by John Barclay Carter on Oct. 18, 2004. This application claims further priority to U.S. provisional patent application, entitled “GRAS Two Acid Low pH Compound consisting of Citric and Hydrochloric Acids,” Ser. No. 60/517,797, filed Nov. 5, 2003 by John Barclay Carter.

### FIELD OF THE INVENTION

The present invention relates generally antimicrobial compositions and specifically to antimicrobial compositions with a low pH that are safe for human consumption.

### BACKGROUND OF THE INVENTION

Low pH compositions and solutions containing such compositions are widely used. They are used in industry, food processing, sterilization and cleaning. Some are dangerously caustic to humans and animals. Some are non-caustic to humans but contain components that are not generally recognized as safe (GRAS) by the U.S. Food and Drug Administration. Others are made of GRAS components but still contain objectionable pollutants.

### SUMMARY OF THE INVENTION

This invention is beneficial for environmental preservation while satisfying the need for a low pH compound. This invention is an environmentally friendly low pH formula for decontamination. It is an anti-microbial agent in the form of a low pH acidic aqueous solution consisting of two acids with a pH of less than one when combined. The embodiment includes the Monobasic Hydrochloric Acid and the Polybasic Citric Acid. The invention is non-toxic to humans and animals. The disposal of this invention after use is a less objectionable pollutant into the environment. This invention is intended for use as a disinfectant in food production, agriculture, household, cosmetic, medical, industrial, military, public safety, transportation, waste treatment, hygiene, locations of population concentration and institutions of confinement.

This invention provides a low pH solution while accommodating environmental needs. The eutrophication of bodies of water results from the increase of nutrients, such as phosphorus, caused by current products. This causes increases in photosynthetic algae and bacteria, which lowers the water's oxygen level, and causes fish deaths. This invention satisfies the need for a low pH solution which protects bodies of water from these problems. The invention provides a decontaminant and sterilant for homes, offices, industrial facilities, restaurants, medical facilities, military installations, prisons, cruise ships and other places where people congregate. It also provides a decontaminant and sterilant for animal cages and facilities. The present invention is nontoxic and ingestible. Therefore, no protective gear or special skills are required for its use. It can be used in food and drink products. The present invention's residue on food also kills transmittable microbials. It provides room temperature sterilization for health care instruments, thereby reducing burn risks to employees from harsh chemicals and/or autoclaves. Medically, either alone or as an additive, it treats wounds, sexually transmitted diseases, burns, infections and digestive disorders. It kills germs in fabrics without bleaching colors. It kills odors from urine by killing the germs causing the odor rather than masking the odor. It satisfies the industrial needs for a low pH solutions without the risks to employees and customers usually associated with such solutions. It provides a skin wash for personnel after contact with persons, animals or items that may be infectious. Because it is nontoxic, it can be shipped in high concentration and diluted to the required level at its destination, thereby reducing shipping costs in labor, fuel and traffic congestion. Insurance costs should be reduced because an accidental spill would not be a catastrophic danger to the public. The invention is substantially noncorrosive to metals. The invention is innocuous to humans, animals, and is safe for use on and in ingestible products. The invention is safe for use on surfaces that contact ingestible products. The solution may be safely used for wound treatment, decontamination, and may be used as a pharmaceutical additive. In the preferred embodiment, the component solutions are admixed to create the present invention in concentrations that reduce the pH of the present invention to less than 1.

The present invention now will be described more fully hereinafter with reference to the accompanying drawings, which are intended to be read in conjunction with both this summary, the detailed description, and any preferred and/or particular embodiments specifically discussed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### DETAILED DESCRIPTION OF THE INVENTION

The present invention is prepared by creating an organic acid solution by dissolving granulated polybasic organic citric acid in water and admixing a monobasic inorganic hydrochloric acid with the citric acid solution. The resulting solution is then stirred until a homogenous consistency is reached. The resulting solution may then be diluted with water until the desired pH is reached. The amount of dilution acceptable is dependent upon the desired pH level and the stringency of the consumer's demand for quality and consistency. As an example, in the preferred embodiment, one U.S. Gallon of the invention with a pH of approximately 0.7 is created by dissolving 8 ounces, by weight, of granulated citric acid into 115 fluid ounces of water and stirring until substantially all of the granulated citric acid is dissolved. 8.5 fluid ounces of hydrochloric acid is then admixed to the citric acid solution and stirred until a homogenous consistency is reached. The pH of the invention may be raised by dilution of the invention with water until a minimal pH level is attained to satisfy the user's needs and reduce the cost of the application. While not meant as limiting, this example produces the preferred embodiment of the invention. One skilled in the art will recognize that many variations in concentration of the components will achieve similar results. Such related concentrations are intended to be and are covered by the present disclosure.

This invention is intended for use alone in full concentration, diluted or as a component of other solutions. In the preferred embodiment, full concentration is 35% hydrochloric acid, 10% citric acid, and 55% water. This invention also relates to the dilution and application methods of this invention.

The applications of this invention include but are not limited to sterilization of surgical locations, sterilization of medical equipment, sterilization of biological waste for sanitary disposal, sterilization and deodorization of veterinary clinics, countermeasure to chemical and biological warfare, disinfectant to speed healing for bum victims, disinfectant to speed healing athlete's foot, sterilization of drinking water, food preservation, household cleaner that is not dangerous to children and pets, glass cleaner, remover of oxidation from a variety of metals, deodorize and sanitize urine stains and pet odor, additive to cosmetics and personal deodorants, sterilization of tattoo parlor equipment, sterilization of manicure equipment, deodorize clothing and shoes, cleaning build up in industrial process and transportation equipment, as a disinfecting rinse for food during processing, sanitizing restaurants both in public and food preparation areas, flushing heating and cooling systems to thwart Legionnaire's Disease, decontamination after a chemical spill, disinfecting for first responders rendering aid to HIV patients, disinfecting public areas during the cold and flu season to reduce absenteeism, control swimming pool pH without dangerous chemicals, paper pulp processing, and breaking down sewage waste resulting in reduced eutrophication.

The aqueous organic acidic solution that is the subject of the present invention may be used for sterilization and microbial growth inhibition purposes. The aqueous organic acidic solution is prepared by dissolving granulated polybasic organic citric acid in water, adding a sufficient amount of monobasic inorganic hydrochloric acid to reduce the pH to of the solution to a value of less than two, and mixing the solution until a homogenous consistency is reached. The amounts of the ingredients used effects the pH of the resultant solution. As the solution is diluted by water, the pH value is increase. Higher pH value solutions are therefore cheaper to produce but are less effective when used for sterilization and microbial growth inhibition. In the preferred embodiment, the pH value of the solution is below 1.

As discussed above, the present invention may be use to sterilize and inhibit microbial growth on food products during processing. The method of sterilizing and inhibiting microbial growth comprises obtaining an aqueous organic acidic solution having a pH value below 1 and consisting of aqueous organic citric acid and monobasic inorganic hydrochloric acid. The solution may be obtained by manufacture of the solution or purchase from another party. The method further comprises adding sufficient water to the aqueous organic acidic solution to create an aqueous acidic rinse. Finally, the aqueous acidic rinse should be applied directly to the food products being processed. The aqueous acidic rinse may also be applied to food preparation surfaces, food processing machinery, food transportation equipment, and any other locations where food may come in contact with contaminants. One of ordinary skill in the art will understand that the aqueous acidic rinse may be applied in many ways including, but not limited to, included with the food product as an additive, immersion, flush, spray, and transfer application. As mentioned above, the amount of water added to the aqueous organic acidic solution to create the aqueous acidic rinse effects the pH value, costs, and effectiveness of the rinse. Adding more water to the aqueous acidic rinse reduces the cost of the rinse by volume, but increases the pH value and reduces the effectiveness of the rinse.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification.

1-13. (canceled)

14. A method of sterilizing and inhibiting microbial growth on a food product comprising:
providing an aqueous acidic solution consisting essentially of citric acid and hydrochloric acid with a pH of less than 2; and
applying the aqueous acidic solution to the food product.

15. The method of claim 14, wherein the aqueous acidic solution consists of citric acid and hydrochloric acid.

16. The method of claim 14, wherein the step of applying comprises applying the aqueous acidic solution to the surface of the food product.

17. The method of claim 14, wherein the pH of the aqueous acidic solution is less than 1.

18. The method of claim 14, wherein the method does not comprise rinsing the aqueous acidic solution off the food product.

19. The method of claim 14, wherein 1 gallon of the aqueous acidic solution consists of 8 ounces of citric acid, 8.5 fluid ounces of concentrated hydrochloric acid, and 115 fluid ounces of water.

20. The method of claim 14, wherein the aqueous acidic solution is a direct food additive.

21. The method of claim 14, wherein the aqueous acidic solution is safe for human consumption.

22. The method of claim 14, wherein the aqueous acidic solution is antimicrobial.

23. The method of claim 14, wherein the aqueous acidic solution is applied as an additive, immersion, flush, or spray.

24. The method of claim 14, further comprising diluting the aqueous acidic solution with water prior to applying the aqueous acidic solution to the food product.

25. A method of sterilizing and inhibiting microbial growth on a food preparation surface, food processing machinery, or food transportation equipment comprising:
providing an aqueous acidic solution consisting essentially of citric acid and hydrochloric acid with a pH of less than 2; and
applying the aqueous acidic solution to the food preparation surface, food processing machinery, or food transportation equipment.

26. The method of claim 25, wherein the aqueous acidic solution consists of citric acid and hydrochloric acid.

27. The method of claim 25, wherein the pH of the aqueous acidic solution is less than 1.

28. The method of claim 25, wherein the method does not comprise rinsing the aqueous acidic solution off the food preparation surface, food processing machinery, or food transportation equipment.

29. The method of claim 25, wherein 1 gallon of the aqueous acidic solution consists of 8 ounces of citric acid, 8.5 fluid ounces of concentrated hydrochloric acid, and 115 fluid ounces of water.

30. The method of claim 25, wherein the aqueous acidic solution is safe for human consumption.

31. The method of claim 25, wherein the aqueous acid solution is antimicrobial.

32. The method of claim 25, wherein the aqueous acidic solution is applied as an additive, immersion, flush, or spray.

33. The method of claim 25, further comprising diluting the aqueous acidic solution with water prior to applying the aqueous acidic solution to the food preparation surface, food processing machinery, or food transportation equipment.
